(19) United States
(12) Patent Application Publication
(10) Pub. No.: US 2024/0349730 A1
(43) Pub. Date: Oct. 24, 2024

(54) DRIED BIOLOGICAL COMPOSITIONS AND METHODS THEREOF

(71) Applicant: Evonik Operations GmbH, Essen (DE)

(72) Inventors: Mathias DERNEDDE, Ronneburg (DE); Max BRAUN, Rothenburg (DE); Harald SCHMIDT, Mülheim an der Ruhr (DE); Sabine LEICK, Ronneburg (DE)

(73) Assignee: Evonik Operations GmbH, Essen (DE)

(21) Appl. No.: 18/683,134

(22) PCT Filed: Aug. 17, 2022
(86) PCT No.: PCT/EP2022/072888

§ 371 Date: Feb. 12, 2024

(30) Foreign Application Priority Data
Aug. 26, 2021 (EP) 21193146.4

(51) Int. Cl. A01N 63/27; A01N 25/26; A01N 43/16; A01N 59/00; A01P 21/00

(52) U.S. Cl. CPC A01N 63/27; A01N 25/26; A01N 43/16; A01N 59/00; A01P 21/00

(57) ABSTRACT

The present disclosure relates to a dried biological composition, comprising at least one silica, a mixture of polysaccharides and glycoproteins, at least one microbe, at least one sugar or sugar alcohol and a resin. The dried biological composition is produced by the following steps: (a) mixing of at least one silica, mixture of polysaccharides and glycoproteins, at least one microbe and at least one sugar or sugar alcohol in aqueous solution, (b) spray drying the mixture of (a), (c) coating the spray dried mixture (b) with a compound containing a resin in a fluidized bed. The composition is meant for agricultural usage like foliar spray or seed treatments.

### TECHNICAL FIELD

The present disclosure relates generally to dried biological compositions, and methods of making and using the same.

### BACKGROUND

U.S. Pat. No. 8,409,822 (Trevino et al.) discloses compositions for delivering microbes in a dry mode comprising precipitated silica granules having a porous structure and microbes loaded throughout the pores of the precipitated silica granules, wherein the composition is operable to allow for propagation of the microbes within the pores of the precipitated silica granules. Also, U.S. Pat. No. 9,296,989 (Trevino et al.) discloses compositions for delivering living cells in a dry mode comprising an inert carrier substrate having pores, living cells loaded within the pores of the inert carrier substrate and a surface layer disposed on an outer surface of the inert carrier substrate loaded with living cells, wherein the surface layer is permeable to molecules that aid in cell growth of the living cells such that the composition is operable to allow for increased propagation of the living cells within the inert carrier substrate as compared to another composition having an absence of the surface layer. Although the compositions of Trevino et al. are disclosed to be in “dry mode”, they are not in fact dried as the liquids including the living microbes are disclosed to be substantially loaded into the precipitated silica granule pores. The silica in Trevino et al. acts as an absorbent and is loaded with 25-75% of living microbes. At this level of loading, the loaded silica is free flowing defined as being dry to the touch. These compositions are relatively limited in utility because the concentration of the organisms and the water content in the silica are not optimized and will likely result in quick loss in activity as the organisms can still respire.

Various protectants such as sulfoxides, alcohols, monosaccharides, polysaccharides, amino acids, peptides, glycoproteins and other additive agents have been used to protect the microbes from dehydration damage. U.S. Pat. No. 5,360,607 (Eyal et al.) discloses improved, stable, dried, prilled biopesticidal compositions comprising an inert carrier which is capable of supporting fungal growth and promoting conidia sporulation and an entomogenous fungal biomass prepared by submerged fermentation of the fungus, Paecilomyces fumosoroeus isolate. This method, however, uses alginate to encapsulate a natural prill which are subject to variations, particularly moisture content (e.g., water activity (a_{w}) level) which the microbes rely on to survive and respire.

WO 2020/104612 A1 describes a dried biological composition comprising (1) a substrate and (2) micro-organisms loaded onto the surface of said substrate, wherein the composition has a total moisture content of about 0.01 wt. % to about 15 wt. %.

WO 2012/118795 A2 describes a seed coating composition comprising a seed and a specific layer coating.

CN101069499 A describes a method for treating a seed comprising a film forming agent, a coloring agent and an oxygen enriched porous inorganic material.

None of the prior art addresses the problem of maintaining the stability of the microbes while stored in dried form. Therefore, it is an aim of the present invention to provide microbes in dry and stable form in high concentration during storage.

### SUMMARY

This aim is achieved by a dried biological composition comprising at least one silica, mixture of polysaccharides and glycoproteins, at least one microbe, at least one sugar or sugar alcohol and at least one resin.

The silica could be a fumed or precipitated silica, preferably precipitated silica. The precipitated silica preferably has a BET surface area between 10 m²/g and 550 m²/g, more preferably between 200 m²/g and 550 m²/g, still preferably between 480 m²/g and 520 m²/g. The precipitated silica preferably has a particle size d50 between 5 μm and 200 μm, more preferably between 30 μm and 100 μm, still preferably between 40 μm and 60 μm. The precipitated silica preferably has a total moisture content between 1 wt. % and 15 wt. %, more preferably between 5 wt. % and 10 wt. %, still preferably between 6 wt. % and 8 wt %. The precipitated silica preferably has an oil absorption (DOA) between 10 mL/100 g and 500 mL/100 g, more preferably between 200 mL/100 g and 400 mL/100 g, still preferably between 280 mL/100 g and 300 mL/100 g.

The dried biological composition preferably has a BET surface area between 0.1 m²/g and 5 m²/g, more preferably between 0.5 m²/g and 2 m²/g, still preferably between 0.5 m²/g and 1 m²/g. The dried biological composition preferably has a particle size d50 between 5 μm and 700 μm, more preferably between 300 μm and 600 μm, still preferably between 450 μm and 550 μm. The dried biological composition preferably has a total moisture content between 0 wt. % and 15 wt. %, more preferably between 2 wt. % and 10 wt. %, still preferably between 3 wt. % and 6 wt. %. The dried biological composition preferably has a water activity between 0.1 and 0.6, more preferably between 0.15 and 0.5, still preferably between 0.2 and 0.4.

The mixture of polysaccharides and glycoproteins could be gum arabic. Gum arabic could have a concentration in the dried biological composition between 1 wt. % and 30 wt. %, preferably between 5 wt. % and 15 wt. %. Gum arabic could act as protective and could bring benefits in terms of survival rate during spray-drying.

The microbes could be Bacillus subtilis QST713, Pasteuria usage, Beauveria bassiana, Coniothyrium minitans, Chondrostereum purpureum, Paecilomyces lilacinus, Aschersonia aleyrodis, Beauveria brongniartii, Hirsutella thompsonii, Isaria fumosorosea, Isaria sp., Lecanicillium longisporum, Lecanicillium muscarium, Lecanicillium sp., Metarhizium anisopliae, Metarhizium anisopliae var. acridum, Nomuraea rileyi Sporothrix insectorum, Cydia pomonella GV, Phytophthora palmivora, Lagenidium giganteum, Bacillus thuringiensis, Pseudomonas protegens, Bradyrhizobium, Mycorrhiza, Clonostachys rosea, Saccharomyces cerevisiae, Pichia pastoris, Aspergillus niger, Aspergillus oryzae, or Hansenula, Bacillus spp. and Lactobacillus spp., or any combinations thereof, preferably, selected from the group consisting of Bacillus thuringiensis, Pseudomonas protegens, Bradyrhizobium, Mycorrhiza, Clonostachys rosea and any combinations thereof, more preferably Pseudomonas protegens.

The sugar could be trehalose, saccharose, di- or polysaccharides, preferably isomaltulose or palatinose. The sugar alcohol could be isomalt.

The resin could be shellac.

The dried biological composition preferably comprises at least one precipitated silica, mixture of polysaccharides and glycoproteins, at least one microbe, sugar or sugar alcohol and shellac.

The dried biological composition more preferably comprises at least one precipitated silica, gum arabic, at least one microbe, isomalt and shellac.

The dried biological composition most preferably comprises at least one precipitated silica, gum arabic, Pseudomonas protegens, isomalt and shellac.

The dried biological composition can comprise a plasticizer, preferably polyethylene glycol (PEG) or glycerine, more preferably PEG 400.

The dried biological composition preferably comprises 5 wt. %-15 wt. % silica, 5 wt. %-15 wt. % mixture of polysaccharides and glycoproteins, 2 wt. %-8 wt. % microbes, 20 wt. %-30 wt. % sugar or sugar alcohol and 30 wt. %-60 wt. % resin.

The dried biological composition preferably comprises 5 wt. %-15 wt. % precipitated silica, 5 wt. %-15 wt. % gum arabic, 2 wt. %-8 wt.-% microbes, 20 wt. %-30 wt. % sugar or sugar alcohol and 30 wt. %-60 wt. % resin.

The dried biological composition preferably comprises 5 wt. %-15 wt. % precipitated silica, 5 wt. %-15 wt. % gum arabic, 2 wt. %-8 wt. % Pseudomonas protegens, 20 wt. %-30 wt. % isomalt and 30 wt. %-60 wt. % shellac.

The dried biological composition can have a core shell structure, the core comprising silica, a mixture of polysaccharides and glycoproteins, microbes and sugar or sugar alcohol and the shell comprising a resin, preferably shellac.

The dried biological composition can contain 30 wt. %-60 wt. % resin, preferably shellac.

The dried biological composition preferably comprises 5 wt. %-15 wt. % precipitated silica, 5 wt. %-15 wt. % gum arabic, 2 wt. %-8 wt. % Pseudomonas protegens, 20 wt. %-30 wt. % isomalt and 30 wt. %-60 wt. % shellac and the composition has a core shell structure, the core comprising precipitated silica, gum arabic, Pseudomonas protegens and isomalt and the shell comprising shellac.

The process according to the invention comprises
(a) mixing of at least one silica, a mixture of polysaccharides and glycoproteins, at least one microbe and at least one sugar or sugar alcohol in aqueous solution,
(b) spray drying the mixture of (a),
(c) coating the spray dried mixture (b) with a compound containing a resin in a fluidized bed.

The mixing step (a) could be done with at least one silica, a mixture of polysaccharides and glycoproteins, at least one microbe and at least one sugar or sugar alcohol in aqueous solution. The aqueous solution of step (a) could be a physiological saline solution.

The aqueous solution of step (a) could be prepared by
(i) mixing a mixture of polysaccharides and glycoproteins, for example gum arabic, sugar or sugar alcohol, for example isomalt and silica, preferably precipitated silica, for example Sipernat® 50,
(ii) centrifuge microbes, the residue after centrifugation could be suspended in physiological saline solution or the culture medium Luria Broth (Luria-Miller Bertani Broth) and
(iii) after complete suspension, the two solutions (i) and (ii) can be mixed for about 30 minutes to 60 minutes.

The spray-drying process (b) could be done with an inlet temperature of the spray-dryer between 76° C. and 80° C. The outlet temperature could be between 45° C. and 60° C. The two-fluid nozzle using air or inert gas could not build up pressure within the system. The whole process could be conducted under nitrogen atmosphere.

The atomization of the mixture could be done in co-current flow with the hot drying gas from the top in the spray-dryer.

In the fluid bed coating process (c) the process gas could be nitrogen with a maximum bed temperature between 40° C. and 50° C., preferably 40° C. The resin could be an aqueous resin solution. The aqueous resin solution could contain a polyethylene glycol or glycerine. The aqueous resin solution could be sprayed into the fluidized bed. The aqueous resin solution could be a shellac solution, preferably an ammonium-salt-shellac solution. The ammonium-salt-shellac solution could contain 25 wt. % pure shellac. The ammonium-salt-shellac solution could contain PEG 400 (for example 5 wt. % referred to dried shellac) as a plasticizer.

The inventive dried biological composition could be used in agricultural applications, such as seed treatment or foliar spray application and in food and feed applications.

The final dried biological composition has an improved storage stability.

### EXAMPLES

Particle SIZE (d50).

Substrate particle size measurement is conducted on HORIBA Laser Scattering Dry Particle Size Distribution Analyzer LA-950 through the angle of scattered laser light, according to ISO 13220.

### Total Moisture Content.

Dried substrate or microorganism powder moisture measurement is conducted on Satorius IR Moisture Balance. A mass of about 0.3 g of the sample powder is weighed and kept in an aluminum plate, while heating the sample to a temperature of 105° C. to constant weight.

### Water Activity.

Water activity (a_{w}) of samples is carried out by 22° C. Water activity (a_{w}) of samples is measured by placing the sample in a defined water activity environment, which is tracked using a measuring device. The humidity sensor is based on a capacitive polymer humidity-sensing element, which consists of a hygroscopic dielectric material placed between a pair of electrodes. The sensor is using a plastic or polymer as the dielectric material. Gaseous water molecules can pass inside the sensor. When the moisture increases the material reacts and the sensor geometry determines the value of the capacitance, which relates to the amount of water molecules present. The relative humidity is therefore tracked over time within the chamber and therefore within the sample, since the humidity will follow an equilibrium state between the sample and the gaseous environment.

### BET Surface Area and DOA.

The BET surface areas of the substrates (e.g. silica) is determined with a Micromeritics TriStar 3020 instrument by the BET nitrogen adsorption method of Brunaur et al., J. Am. Chem. Soc., 60, 309 (1938), which is known in the field of particulate materials, such as silica and silicate materials. Nitrogen adsorption-desorption isotherms were collected at 77K. Powdered samples of 50-100 mg are degassed at 105° C. for 2 hours prior to measurement. Barrett-Joyner-Halenda (BJH) models are used to calculate BET surface area. DOA absorption measurement is done using ISO 19246.

### Colony Forming Unit (CFU).

The concentration of the microbes is determined by plate count using serial dilution techniques. The product powder containing microbes is stirred in sterile water with Triton X-100 surfactant present to mobilize the microbes from the carrier. The resulting suspension of individualized microbes is sequentially diluted several times, each time by a factor of 10. Each time a sample of the dilution is plated onto sterile agar plates and incubated. After several days the organisms present can be seen as dots on the agar. When the dilution is sufficient to reduce the number on the plate to a countable quantity, the number of colonies is counted and multiplied by the dilution factor to determine the population in the original sample.

### Storage Test

The storage test is done at 25° C. The storage test is conducted in a climate chamber with a salt concentration defining a water activity environment in the surrounding atmosphere of 40% relative humidity (sodium iodide), what is equivalent to a water activity of 0.4. The uncoated samples from the spray-drying process as a reference, as well as the coated samples from the fluid bed coating process are added in 1 g portions into the chambers in open falcon tubes. Over time, the relative humidity in the closed atmosphere will be the same as in the open sample tube with each 1 g sample. The cells therefore equilibrate with the relative humidity of the surrounding atmosphere. Over a given time period (several weeks), samples are drawn from the climate chambers in regular intervals. Samples are then analyzed by the described CFU method.

Using the analytical methods described above, the physical properties of the substrates are measured and summarized below.

### Example 1 (Comparison)

In one flask 6390 g physiological saline solution is provided. This will be called solution (i). 655 g of gum arabic is weighed in and is stirred with a propeller mixer until the gum arabic is completely dissolved. Afterwards 1829 g sugar alcohol Isomalt (Risumalt®) is added to the solution. After complete dissolution 770 g of Sipernat® 50 are given into the liquid and are mixed until homogeneously distributed. Sipernat® 50 is a product of Evonik Operations GmbH with a BET of 500 m²/g, a d50 of 50 μm and total moisture content of ≤7 wt. %.

Bacterial biomass of Pseudomonas protegens Migula, Pf-5 is harvested from an overnight culture in a shaking flask by centrifugation at 12000 rpm for 10 minutes. The harvested biomass cell pellet, which contains the microbes, is transferred to a second flask. 1600 g of these microbes are re-suspended in 1600 g of a fresh Luria Broth (Luria-Miller Bertani Broth) culture medium, using a propeller mixer. This solution is referred to as solution (ii).

Solution (i) is given into solution (ii) and stirred additionally 60 minutes. This solution is referred to as solution (iii).

Solution (iii) is spray-dried using a Niro Minor (MM-100) Spray Dryer manufactured by GEA. The inlet temperature is adjusted to 80° C. with a flow rate of 18 g/min to 20 g/min. The corresponding outlet temperature is between 50° C. and 53° C. The dryer is used in a co-current flow and the particles atomized using a two-fluid nozzle are dried within seconds inside the machine. The atomization and drying gas is nitrogen. A cyclone is collecting the produced particles from where the product is then stored cool at 4° C.

### Example 2 (Inventive Example)

2.4 kg spray-dried product of example 1 are stored inside the fridge at 4° C. until they are used the next day in a fluid bed called Procell LabSystems, manufactured by Glatt. In one run, 350 g of spray-dried product of example 1 is provided inside the fluid bed machine. The 30 m³/h nitrogen used to fluidize the powder is preheated to 80° C., before 1475 g ammonium-shellac solution (SSB® AQUAGOLD of Stoever GmbH & Co. KG Bremen) is sprayed into the fluidized powder bed, using a 1.5 bar nozzle pressure. The aqueous ammonium-shellac solution has a concentration of 25 wt % dry shellac in water. A plasticizer is added to this solution in a concentration of 5 wt % according to the dry shellac weight used. The plasticizer is PEG 400. The drying time is 140 minutes. The bed temperature is about 40° C. The shellac will form a shell around the spray-dried particles and coat them accordingly. The coated powder contains shellac on the outside.

Using the methods described or similarly described above, the BET surface area and the mean particle size of the products directly after the processes are measured and summarized in Table 1.

TABLE 1

| | | BET Surface | Particle size |
|---|---|---|---|
| Substrates | Process | Area [m²/g] | d50 [μm] |
| Example 1 | Spray-Dried | 20.48 | 60 |
| Example 2 | Fluid Bed coated | 00.73 | 500 |

Using the methods described or similarly described above, the total moisture content and water activity (a_{w}) level of the products directly after the processes are measured and summarized in Table 2.

TABLE 2

| | | Total Moisture | Water Activity |
|---|---|---|---|
| Substrate | Process | Content [wt %] | (a_{w}) [—] |
| Example 1 | Spray-Dried | 4.0 | 0.33 |
| Example 2 | Fluid Bed coated | 4.2 | 0.38 |

The storage water activity (a_{w}), which describes the water activity of the atmosphere the samples are stored in, storage temperature (° C.), initial CFU and the moisture content effect on CFU after one to three weeks is summarized in Table 3.

TABLE 3

| | | | | | | | LOG Loss |
|---|---|---|---|---|---|---|---|
| | | | | | CFU/g | CFU/g | between |
| | | Storage | | | biodrymass | biodrymass | initial CFU |
| | | Water | Storage | Initial | after storage | after storage | and storage |
| | | Activity | Temp. | CFU/g | after one | after three | after three |
| Substrate | Process | (a_{w}) [—] | [° C.] | biodrymass | week | weeks | weeks |
| Example 1 | Spray- | 0.40 | 25° C. | 8.80E+11 | 1.02E+07 | 3.04E+04 | 7.46 |
| | Dried | | | | | | |
| Example 2 | Fluid | 0.40 | 25° C. | 3.08E+09 | 5.97E+07 | 2.50E+06 | 3.09 |
| | Bed | | | | | | |
| | coated | | | | | | |

As can be seen from the table 1 above, the BET surface decreases due to the blockage of the open pores, by addition of a shellac coating.

At 40% RH the uncoated powder (example 1) shows a lower stability compared to the shellac coated powder (example 2) at the same storage condition (table 3). After three weeks the coated material has a LOG Loss of 3.09, the uncoated material a LOG loss of 7.46.

Therefore, it can be concluded, that a shellac coating decreases the death rate of the alive cells during storage significantly and protects the cells from dying, compared to the uncoated material.

1. A dried biological composition comprising at least one silica, mixture of polysaccharides and glycoproteins, at least one sugar or sugar alcohol, at least one microbe and at least one resin.

2. A dried biological composition according to claim 1, wherein the silica is a precipitated silica.

3. A dried biological composition according to claim 2, wherein the precipitated silica has a BET surface area between 10 m²/g and 550 m²/g, preferably between 200 m²/g and 550 m²/g, more preferably between 480 m²/g and 520 m²/g.

4. A dried biological composition according to claim 1, wherein the microbe is Bacillus subtilis QST713, Pasteuria usagae, Beauveria bassiana, Coniothyrium minitans, Chondrostereum purpureum, Paecilomyces lilacinus, Aschersonia aleyrodis, Beauveria brongniartii, Hirsutella thompsonii, Isaria fumosorosea, Isaria sp., Lecanicillium longisporum, Lecanicillium muscarium, Lecanicillium sp., Metarhizium anisopliae, Metarhizium anisopliae var. acridum, Nomuraea rileyi Sporothrix insectorum, Cydia pomonella GV, Phytophthora palmivora, Lagenidium giganteum, Bacillus thuringiensis, Pseudomonas fluorescens, Bradyrhizobium, Mycorrhiza, Clonostachys rosea, Saccharomyces cerevisiae, Pichia pastoris, Aspergillus niger, Aspergillus oryzae, or Hansenula, Bacillus spp. and Lactobacillus spp., or any combinations thereof, preferably, selected from the group consisting of Bacillus thuringiensis, Pseudomonas protegens, Bradyrhizobium, Mycorrhiza, Clonostachys rosea and any combinations thereof.

5. A dried biological composition according to claim 4, wherein the microbe is Pseudomonas protegens.

6. A dried biological composition according to claim 1, wherein the resin is shellac.

7. A dried biological composition according to claim 1, wherein the sugar or sugar alcohol is trehalose, saccharose or polysaccharide or isomalt.

8. A dried biological composition according to claim 1, comprising precipitated silica, gum arabic, Pseudomonas protegens, isomalt and shellac.

9. A dried biological composition according to claim 1, comprising 5 wt. %-15 wt. % silica, 5 wt. %-15 wt. % mixture of polysaccharides and glycoproteins, 2 wt. %-8 wt. % microbes, 20 wt. %-30 wt. % sugar or sugar alcohol and 30 wt. %-60 wt. % resin.

10. A dried biological composition according to claim 1, wherein the composition has a core shell structure, the core comprising silica, a mixture of polysaccharides and glycoproteins, microbes and sugar or sugar alcohol and the shell comprising a resin.

11. A dried biological composition according to claim 10, wherein the composition contains 30 wt. %-60 wt. % resin.

12. A dried biological composition according to claim 10, comprising 5 wt. %-15 wt. % silica, 5 wt. %-15 wt. % a mixture of polysaccharides and glycoproteins, 2 wt. %-8 wt. % microbes, 30 wt. %-60 wt. % resin and 20 wt. %-30 wt. % sugar or sugar alcohol.

13. Process for the production of the dried biological composition according to claim 1 comprising the steps of:
(a) mixing of at least one silica, mixture of polysaccharides and glycoproteins, at least one microbe and at least one sugar or sugar alcohol in aqueous solution,
(b) spray drying the mixture of (a),
(c) coating the spray dried mixture (b) with a compound containing a resin in a fluidized bed.

14. Process for the production of the dried biological composition according to claim 13, adding physiological saline solution in step (a).

15. Process for the production of the dried biological composition according to claim 13, adding glycerine or polyethylenglycol in step (c).

16. Use of the dried biological composition according to claim 1 for seed formulations or foliar spray applications.
